(19) United States
(12) Patent Application Publication
(10) Pub. No.: US 2005/0100172 A1
(43) Pub. Date: May 12, 2005

(54) Method and arrangement for processing a noise signal from a noise source

(76) Inventors: Michael Schliep, Aichtal (DE); Szabolcs Toergyekes, Stuttgart (DE); Walter Zipp, Stuttgart (DE)

Correspondence Address: CROWELL & MORING LLP;INTELLECTUAL PROPERTY GROUP, P.O. BOX 14300, WASHINGTON, DC, 20044-4300, US

(21) Appl. No.: 10/451,416

(22) PCT Filed: Dec. 12, 2001
(86) PCT No.: PCT/EP01/14622

(30) Foreign Application Priority Data
Dec. 22, 2000 (DE) 100-64-754-5

(51) Int. Cl. A61F 11/06; G10K 11/16; H03B 29/00; H04R 29/00

(52) U.S. Cl. 381/71.4; 381/57; 381/56

(57) ABSTRACT

For a reliable identification of noise sources generating noise or sound signals, according to the invention, in a method for determining a noise signal (S) of a noise source (10), the noise signal (S) is detected and analyzed on the basis of signal properties, the noise signal (S) being compared with noise patterns (M) and being assigned to a noise source type (T) on the basis of the comparison.

### BACKGROUND AND SUMMARY OF THE INVENTION

This application claims the priority of German patent document 100 64 754.5, filed 22 Dec. 2000 (PCT International Application No. PCT/EP01/14622, filed 12 Dec. 2001), the disclosure of which is expressly incorporated by reference herein.

The present invention relates to a method and apparatus for determining a noise signal of stationary or moveable noise sources, such as a vehicle.

In order to comply with legal noise limit values in the case of, for example, an aircraft taking off or landing, or a passing vehicle, noise reduction measures on the vehicle are available to improve the traffic noise affecting the surroundings and traveling comfort. For example, low-noise exhaust gas and intake systems, largely resonance-free propulsion units, sound-absorbing bodywork are known for sound reduction purposes for vehicles, such as motor vehicles, rail vehicles or aircraft. However, the noise reduction measures on the vehicle and the resulting reduction of the noise level are limited. At present, measures or ambient conditions that influence the noise level (such as a low-noise highway or ambient meteorological conditions) are not taken into account in gauging complying with noise limit values.

It is customary to provide stationary, passive measuring devices to detect and monitor emission values for quantities such as benzene and particulars. In addition, sound emissions occurring at the same location can also be measured, if appropriate. However, such passive, location-related sound emission measurement is not suitable for identifying the noise sources from which the noise emanates. Moreover, noise reduction measures, over and above the measures on the vehicle, are not possible.

Japanese patent document JP 05081595 A describes a method for identifying vehicle types on the basis of the engine noise that they generate. For this purpose, measured noises are compared with noise patterns stored in a memory.

One object of the present invention to provide a simple and reliable method and apparatus for determining a noise signal of a noise source.

This and other objects and advantages are achieved by the noise detection and processing method according to the invention in which the noise signal is detected and analyzed on the basis of specified signal properties. The noise signal is compared with noise stored patterns, and is assigned to a noise source type on the basis of the comparison. Such an analysis (particularly a time and/or frequency analysis) of signal properties of the detected noise signal and the assignment thereof to the type of underlying noise source enables a documentation of temporal and/or spatial behavior of the noise source. Alternatively, or in addition, open- and/or closed-loop noise abatement measures can be implemented on the basis of the noise signal thus determined and the underlying noise source type.

The invention is based on the proposition that, in order to comply with noise limits, (e.g., in residential areas, in the vicinity of hospitals or in factory buildings), sound emissions should be detected and monitored, not only as a local variable, but also noise source accounting for such sound emissions should be determined. To that end, the detected noise signal (in particular the amplitude and/or frequency values thereof) is analyzed and assigned to an underlying noise source, based on predetermined noise patterns.

Preferably, amplitude values and/or frequency values of the noise signal are evaluated as signal properties. Such a temporal and/or spatial analysis of the signal properties of the noise signal enables an assessment of the noise and/or disturbance levels and a classification thereof for the relevant noise source. For example, a movement of the noise source can be detected on the basis of chronologically detected noise signals source emanating therefrom, and the analysis thereof. To that end, the noise signal is preferably corrected on the basis of a frequency analysis taking account of the acoustic Doppler effect in accordance with the following relationships:

| Noise source | Observer | Observed frequency |
|---|---|---|
| • | [symbol] | f_{B} = f_{Q} • (1 + v_{B}/c) |
| • | •→ | f_{B} = f_{Q} • (1 − v_{B}/c) |
| •→ | • | f_{B} = f_{Q} • (1 − v_{Q}/c) |
| [symbol] | • | f_{B} = f_{Q} • (1 + v_{Q}/c) |
| •→ | [symbol] | f_{B} = f_{Q} • (c + v_{B})/(c − v_{Q}) |
| [symbol] | •→ | f_{B} = f_{Q} • (c − v_{B})/(c + v_{Q}) |
| [symbol] | [symbol] | f_{B} = f_{Q} • (c + v_{B})/(c + v_{Q}) |
| •→ | •→ | f_{B} = f_{Q} • (c − v_{B})/(c − v_{Q}) |

where f_{B}=frequency perceived by the observer (that is, the frequency detected by a noise sensor), f_{Q}=frequency of the noise source, V_{B}=velocity of the observer, V_{Q}=velocity of the noise source, c=speed of sound.

Alternatively, for a stationary noise source, such as an electric motor in a production building, it is possible to classify the recorded noise signals of airborne or structure-borne sound, to identify operational faults or operating states (e.g., start-up of the electric motor), on the basis of the assessment of the amplitude and accordingly on the basis of the noise and disturbance level and the comparison thereof with noise patterns.

Advantageously, the noise source is optically detected and analyzed, enabling a qualified evaluation of the noise source type. This in turn enables an unambiguous assignment of the noise signal to a model of the noise source type, for example the “A Class” model in the case of a vehicle or the “lathe” or “cutter” model in the case of a machine. A more accurate assignment of noises to noise sources is thus made possible.

For assignment of noise signals from a moving source, it is preferable to determine the movement of the noise source, and to correct the noise signal therefrom on the basis of such movement. Such correction makes it possible to identify the noise source type, (e.g., road or rail vehicle type or the aircraft type). To that end, acoustic analysis of such noise signals is preferably combined with a speed analysis, which enables conclusions to be drawn with regard to movement and/or acceleration states of the noise source (e.g., a vehicle). Alternatively, or in addition, it is possible to determine interactions (particularly, acoustic interactions) with the surroundings, which result from the movement of the noise source.

In an advantageous embodiment, at least one factor acting on the noise source is determined, and the noise signal resulting from the noise source is corrected on the basis of such factor. For example, climate conditions (e.g., rain, temperature, atmospheric humidity, wind) are determined as factors affecting the noise source. As a result, such disturbance signals which influence the noise signals are attenuated or eliminated entirely, for example, in the event of an assignment of noise signals to a noise source type in free surroundings and thus in an open state. The noise source type can thus be identified as accurately as possible. In particular, during an evaluation of the disturbance signals comprising the noise signals, it is possible to draw conclusions about instantaneous operating conditions (e.g., heavy rain), or about functional or operational faults (e.g., severe humming noise in the case of a motor).

Expediently, position and/or ambient conditions of the noise source are determined, and the noise signal is corrected accordingly. By taking account of the location and location-related conditions (e.g., absorption and reflection conditions) in the surroundings, it is possible to correct the noise signal with regard to nonsteady-state absorption and reflection conditions caused by movement of the noise source. The noise signal is preferably stored in a data memory. Predictive or retrospective acoustic analyses and/or statistics of noise signals, such as operating noises of stationary objects (such as motors in a production building), or of moving objects (such as vehicles) are made possible on the basis of the noise signals stored chronologically in the data memory and any detected external parameters, such as climate parameters and location parameters. In this case, different noise patterns are stored for different types of vehicles under different conditions in the data memory. Depending on type and embodiments, these noise patterns are updated and extended on the basis of the currently detected noise signals and the assignment thereof to a noise source type. In this case, the extension of the database for the noise patterns encompasses both climate, location-related, type-related changes and the effects thereof on the sound or noise signal issuing from the noise source.

The noise signal assigned to a noise source type can be used for the open- and/or closed-loop control of noise-reducing systems. To that end, noise signals that have been detected and, if appropriate, corrected on the basis of detected external parameters, are fed to an external system for open- and/or closed-loop control (e.g., for noise-reducing load control of a vehicle or for emergency control of an object in the event of identified functional, material or operational faults). On the basis of the determined data (noise signals and/or external parameters) and analyses or statistics resulting therefrom, the external system serves for open- and/or closed-loop control, information and/or warning particularly for noise reduction in road traffic, for example by influencing the traffic routing). In other words, in the event of an increased volume of traffic and thus a very high noise intensity in the road traffic (e.g., in a residential area), which is detected and analyzed on the basis of the noise signals detected, a corresponding control of the road traffic for noise reduction purposes is implemented in conjunction with a traffic guidance system that may be present or a light signal open-loop/closed-loop control.

As an alternative, the system can be used for tracking identified noise sources over a regional area. The value determined for the object-related noise signal can be fed to an information system of the object (e.g., a vehicle), or the value determined for the weather-adjusted noise signal can be fed to a navigation system.

Preferably, an operating noise of a vehicle is detected as a noise signal, and the vehicle's type, its movement state and/or its acoustic influence on its surroundings are determined by analysis of the noise signal in connection with a speed and model analysis of the vehicle. For example, a corresponding signal from a central system for setting a noise-reduced journey of the vehicle can be fed to a noise-reducing system for load control present in the vehicle.

The apparatus according to the invention, for determining a noise signal of a noise source, includes a noise detection system (advantageously, a plurality of noise sensors) for detecting the noise signal, and a data processing unit for analyzing it on the basis of its signal properties, comparing its signal with noise patterns, and assigning it to a noise source type on the basis of the comparison. Preferably, a network of noise sensors (e.g., direction-sensitive sensors) is distributed along travel routes within towns or in a production or machinery building. For areal detection of the noise signal (particularly in noise-critical areas such as residential areas, in the vicinity of hospitals or in machinery buildings), and thus to identify the varying noise level at different locations, the noise signals detected by the network of sensors can be fed to the central data processing unit if appropriate, for an analytical correction (e.g., taking account of the acoustic Doppler effect, climate influences and/or nonsteady-state absorption and reflection properties).

The data processing unit expediently includes a database of noise patterns for different objects, (e.g., moveable objects, such as road vehicles, rail vehicles, aircraft, or for stationary objects, such as motors or machines in production buildings), if appropriate taking into account different locations, different climate conditions and/or movement of the noise source. On the basis of the noise patterns stored in the database, the noise source type can be identified in a particularly simple and reliable manner, taking account of signals influencing the noise signal.

Advantageously, a data memory is provided for storing the noise signal, for active continuous monitoring and analysis of the noise loading at a location or along a section. Values of the noise signal are stored in the data memory, and thus archived chronologically, (for example, in the form of tables). Depending on the type and functionality of the data processing unit, the chronologically stored noise levels of the noise signal serve for analyses and statistics, in particular for noise statistics. For example, representations relating to the temporal and/or spatial behavior of noises and noise sources and representation relating to the noise loading can be output on the basis of the stored data.

An optical system, for example a video camera, for detecting the noise source is expediently provided to record the location at which at least one noise sensor is arranged. The optical detection system enables, for example, speed analysis of a moving object, which, combined with the noise detection system, provides a combined evaluation of speed and a resultant development of noise of the relevant object such as a vehicle. Furthermore, the speed analysis provides a correction of the acoustic noise signal of the moving object, by taking account of the acoustic Doppler effect. As an alternative or in addition, induction loops, for example, which are arranged along a highway or along a section to be observed, are provided for speed analysis of a relevant moving object.

A further preferred embodiment includes a recording unit for detecting meteorological data, such as temperature, humidity, wind, atmospheric stratification, rain, etc. These data are fed to the central data processing unit so that they can be taken into account in determination of the noise signal, particularly, for the assignment of the noise signal to noise source type.

In the method and apparatus according to the invention, for permanent monitoring of sound and noise emissions, and for reliable identification of noise sources, a noise signal is detected and analyzed on the basis of its signal properties in such a way that a noise source type can be determined and classified on the basis of a comparison with stored noise patterns. Such a categorization of noise sources (e.g., a humming machine in a motor works or a high volume of road traffic), permits use of the arrangement both in closed spaces (e.g., in workshops or production buildings) and in the open surroundings (e.g., along a freeway). In this case, the detected data can be used to make statements about the steady-state, cyclic or nonsteady-state behavior of noise sources in a particularly simple manner.

Other objects, advantages and novel features of the present invention will become apparent from the following detailed description of the invention when considered in conjunction with the accompanying drawings.

### BRIEF DESCRIPTION OF THE DRAWINGS

FIG. 1 shows schematically an arrangement for determining a noise signal of a noise source with a noise detection system and a data processing unit;

FIG. 2 shows schematically the arrangement in accordance with FIG. 1, with an optical detection system for use in road traffic; and

FIG. 3 shows schematically the arrangement in accordance with FIG. 1 for use in a production building.

### DETAILED DESCRIPTION OF THE DRAWINGS

Mutually corresponding parts are provided with the same reference symbols in all the figures.

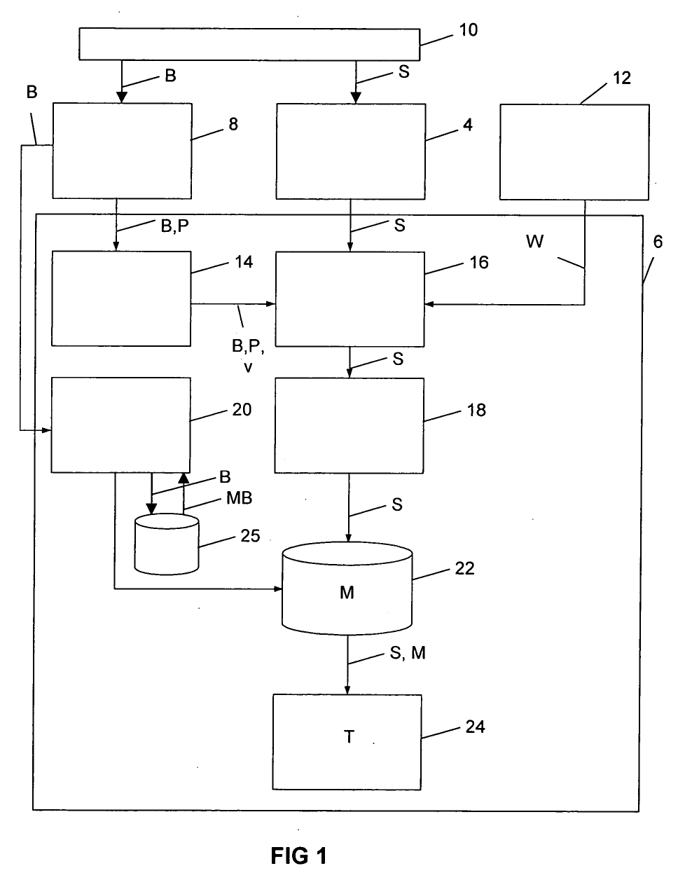

FIG. 1 shows schematically an arrangement 1 for determining a noise signal S with a noise detection system 4 for detecting the noise signal S and with a data processing unit 6 for analyzing the noise signal S on the basis of signal properties, and for comparing the noise signal S with stored noise patterns M. The noise signal S is assigned to a noise source type T on the basis of the comparison.

Also provided is an optical system 8 for recording an image B of a noise source 10 generating the noise signal S and/or a recording unit 12 for detecting meteorological data W. The data processing unit 6 comprises an analysis unit 14 for determining a movement of the noise source 10, in particular for determining the velocity v or the acceleration of the noise source 10, on the basis of the image B detected by the optical system 8. As an alternative, a measurement signal from induction loops (not shown) can be fed to the analysis unit 14 to determine the velocity v. A correction unit 16 is provided to correct for the Doppler effect—resulting from a moving noise source 10—in the sound or noise signal S. On the basis of the movement determined (velocity v or acceleration) the noise signal S generated by the noise source 10 is corrected correspondingly by the correction unit 16. The resulting noise signal S is comparable with measurements on a stationary rolling test bed for vehicles.

Furthermore, the meteorological data W of the recording unit 12 are fed to the correction unit 16 as factors affecting the noise source 10. Such data W are taken into account in the determination of the noise signal S by the correction unit 16. In other words: the noise signal S is correspondingly corrected on the basis of detected climate values, such as temperature, humidity, wind and atmospheric stratification.

The instantaneous position P of the noise source 10 is fed to the correction unit 6 by the optical detection system 8 or another external system (not shown), such as a locating or navigation system. Conditions which influence the noise signal S (e.g., absorption and reflection conditions) in the direct vicinity of the noise source 10 are determined on the basis of the information about the instantaneous position P. The relevant absorption and reflection conditions are taken into account in the determination of the noise signal S.

The corrected noise signal S is fed to an evaluation unit 18, which determines the signal properties of the corrected noise signal S (e.g., based on amplitude and/or frequency values), and in the case of a moving noise source 10 such as a vehicle, the ignition frequency, acceleration and/or the speed thereof). Furthermore, a recognition unit 20 is provided for recognizing the model MO of the noise source type T, (that is, in particular, a vehicle model), on the basis of the detected image B. The recognition unit 20 accesses a database 25 in which image patterns for objects or noise sources 10 are stored. In this case, the pattern library of the database 25 can be updated and extended on the basis of new images of objects or noise sources 10.

In order to determine the noise source type T, the data processing unit 6 comprises a database 22 which includes (depending on its type and scope) a multiplicity of different noise patterns M for the noise signal S of the relevant noise source type T. Such noise patterns M may be purged of factors that influence the noise signal S (e.g., meteorological data W and nonsteady-state absorption and reflection conditions in the surroundings, caused by the movement of the noise source 10). On the other hand, the noise patterns M may be stored without correction, for comparison with the currently detected, uncorrected noise signal S. The data processing unit 6 comprises a comparison unit 24 for this purpose. On the basis of the comparison of the noise signal S that has been detected and, if appropriate, corrected to eliminate influencing factors with the stored noise patterns M, the relevant noise signal S is assigned to the associated noise source type T. For example, in the case of a vehicle as noise source 10, the recognition unit 20 determines the vehicle model (e.g., Mercedes-Benz' C Class), and determines on the basis of the comparison unit 24 determines the motorization of the identified vehicle model and, accordingly, the noise source type T (e.g., Mercedes-Benz' CDI engine) for the noise signal S.

Another example is explained below: if a vehicle representing the noise source 10 has a 4-cylinder, spark-ignition engine and moves at constant velocity v (and thus with a constant engine speed of, for example, 3 000 min⁻¹), then, inter alia, the orifice of the exhaust-gas system emits a humming noise signal S dominated by the ignition frequency of the engine. At the aforementioned 3 000 min⁻¹ (=50 Hz), the 2nd engine order is established as ignition frequency at a frequency of 100 Hz.

A stationary observer or the noise detection system 4 (e.g., a microphone) perceives this humming noise signal S of 100 Hz, as the vehicle drives past, in the form of a rising, then falling frequency on account of the acoustic Doppler effect. If the stationary observer wishes to deduce the frequency-determining engine speed on the basis of a frequency analysis of the humming noise S detected by the microphone 4, he employs the frequency correction equations. To that end, based on a frequency analysis in accordance with the table below for different cases of movement (noise source 10/observer 4), the correction unit 16 takes into account the acoustic Doppler effect resulting therefrom in the determination of the noise signal S. The different possibilities for movement of noise source 10 and observer 4 are indicated by arrows in the aforementioned table. In this case, the velocity of the noise source 10 is designated by v_{Q}, the velocity of the observer 4 is designed by v_{B}, and the speed of sound is designated by c. When employing the formula from the table, the magnitudes of V_{Q}, V_{B} and c are to be inserted into the equations.

| Noise | | |
|---|---|---|
| source 10 | Observer | Observed frequency |
| • | [symbol] | f_{B} = f_{Q} • (1 + v_{B}/c) |
| • | •→ | f_{B} = f_{Q} • (1 − v_{B}/c) |
| •→ | • | f_{B} = f_{Q} • (1 − v_{Q}/c) |
| [symbol] | • | f_{B} = f_{Q} • (1 + v_{Q}/c) |
| •→ | [symbol] | f_{B} = f_{Q} • (c + v_{B})/(c − v_{Q}) |
| [symbol] | •→ | f_{B} = f_{Q} • (c − v_{B})/(c + v_{Q}) |
| [symbol] | [symbol] | f_{B} = f_{Q} • (c + v_{B})/(c + v_{Q}) |
| •→ | •→ | f_{B} = f_{Q} • (c − v_{B})/(c − v_{Q}) |

Consequently, such a combined speed and noise analysis enables conclusions to be drawn about movement and/or acceleration states of the moving noise source 10 of a vehicle. Depending on the type and embodiment of the functionality of the data processing unit 6, the data detected by means of the arrangement 1, such as the noise signal S, the corrected noise signal S, the meteorological data W, the noise source type T, the image B, can be fed to an external open- and/or closed-loop control system (e.g., a load control system of a vehicle for noise-reducing travel, a traffic guidance system for noise-reduced traffic routing, or an open-loop and/or closed-loop control or alarm system of a rotary machine in a production building).

Depending on the type and embodiment of the database 22, the latter serves as a data memory for storing the currently detected data (e.g., the detected noise signal S or the meteorological data W. As an alternative or in addition, a further data memory may be provided. Analyses and statistics, e.g., noise statistics, are made possible on the basis of the stored data, in particular the chronologically detected and stored noise signals S.

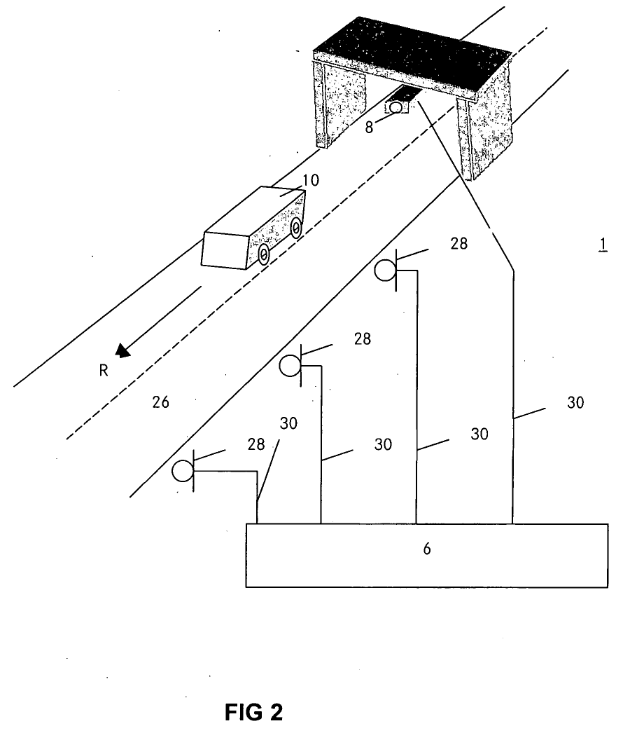

FIG. 2 shows schematically the system of FIG. 1, including the noise detection system 4, with the plurality of noise sensors 18 (which may be direction sensitive microphones) arranged along a highway 26. The noise sensors 28 are connected to the central data processing unit 6 by means of a data transmission unit 30 (e.g., a data bus or a radio link). For detecting an image of the noise source 10, such as a vehicle traveling in the direction R on the highway 26, the optical detection system 8 (e.g., a video camera), is arranged beneath a bridge 32, and is connected to the central data processing unit 6 via the data transmission unit 30.

During the operation of the data processing unit 6, the vehicle or the moving noise source 10, traveling at 50 km/h, for example, is detected by the optical detection system 8 in the form of an image B. Based on the recording image sequence B, by means of the data processing unit 6 determines the velocity v and the noise signal S resulting therefrom, taking account of the acoustic Doppler effect resulting from the movement of the vehicle 10. To that end, the noise signals S detected by the noise sensors 28 undergo a frequency correction in accordance with the acoustic Doppler effect. Furthermore, it is possible to determine the ignition frequency and the overtones thereof (4th, 6th, 8th, etc. engine orders) on the basis of the temporally and spatially detected noise signal S. Alternatively, the velocity v of the vehicle 10 can be determined by means of an induction loop system (not shown) in the highway 26. On account of the ratio of the detected frequencies of the noise signals S to the traveled velocity v, a discrete selection criterion is generated which, together with the vehicle type information, detected by video analysis, and the known transmission ratios of the vehicles underway, permits an unambiguous determination of the vehicle motorization and, accordingly, of the noise source type T.

Depending on the type and embodiment of the arrangement 1, the recording unit 12 can additionally detect meteorological data W, which are taken into account in the correction of the noise signals S detected by the noise sensors 28. Furthermore, the detected data (the detected and, if appropriate, corrected noise signal S which is generated by the movement or by the driving past of the vehicle 10) can be fed to an open-loop and/or closed-loop control system of the vehicle 10 for noise reduction purposes. Alternatively, the data determined by means of the central data processing unit 6, (e.g., the noise signals S detected along the highway 26) may serve for traffic control purposes. For example, a high noise intensity caused by a high volume of traffic which overshoots the permissible sound emission limit value in the relevant area, is determined on the basis of the analysis of the noise signals S. This information can be fed for example to a traffic guidance system for speed restriction purposes or for diverting the road traffic, thereby effecting a noise reduction in this area.

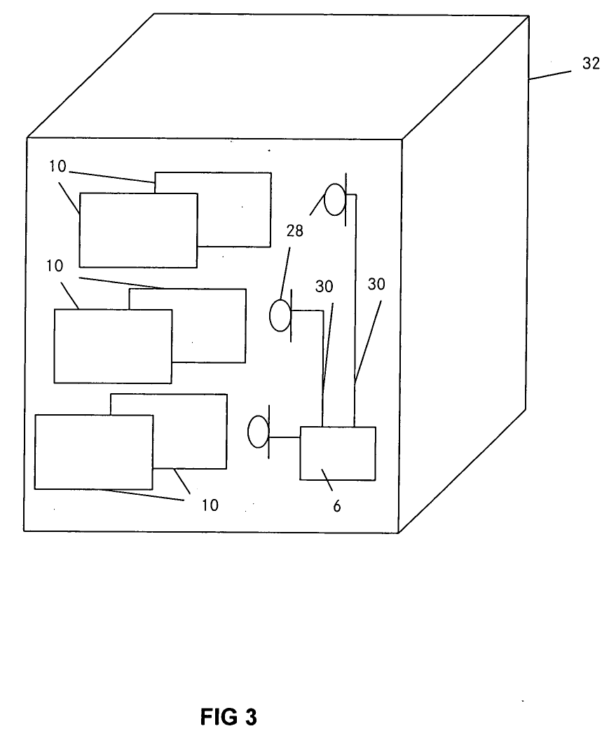

FIG. 3 shows an alternative embodiment of the arrangement 1, for determining the noise signal S in a closed space 30, e.g., in a production building or machinery building. An identification of defective or noisily running machines or motors 10 is made possible on the basis of the noise signals S that have been detected by means of the noise sensors 28 and communicated on the basis of the data transmission unit 30. To that end, the noise signal S is, if appropriate, corrected or purged of disturbance signals analogously to the method described above in road traffic. The noise signal S is compared, on the basis of the data processing unit 6, with the noise patterns M characterizing the machines or motors 10. An assignment of the noise signal S to one of the machines or motors 10 and thus an identification of the defective machine 10 or of incorrect working material and/or an incorrect tool are made possible on the basis of the comparison.

The foregoing disclosure has been set forth merely to illustrate the invention and is not intended to be limiting. Since modifications of the disclosed embodiments incorporating the spirit and substance of the invention may occur to persons skilled in the art, the invention should be construed to include everything within the scope of the appended claims and equivalents thereof.

1-20. (canceled)

21. A method for determining a noise signal of a noise source, comprising:
detecting and analyzing the noise signal on the basis of signal properties thereof;
comparing the noise signal with stored noise patterns; and
assigning the noise signals to a noise source type based on a result of comparison; wherein,
at least one of position and location-related ambient conditions of the noise source are determined; and
the noise signal is corrected on the basis of said at least one of position and location related ambient conditions.

22. The method as claimed in claim 21, wherein at least one of amplitude values and frequency values of the noise signal are evaluated as signal properties.

23. The method as claimed in claim 21, further comprising optically detecting and analyzing the noise source.

24. The method as claimed claim 21, wherein:
a movement of the noise source is determined; and
the noise signal resulting from the noise source is corrected on the basis of the movement.

25. The method as claimed in claim 21, wherein:
at least one factor acting on the noise source is determined; and
the noise signal from the noise source is corrected on the basis of said factor.

26. The method as claimed in claim 21, wherein the noise signal is stored in a data memory.

27. The method as claimed in claim 21, further comprising:
using the noise signal assigned to a noise source type for open- or closed loop control of a noise-reducing system.

28. The method as claimed in claim 21, wherein:
an operating noise of a vehicle is detected as a noise signal; and
at least one of vehicle type, movement state and acoustic influencing of surroundings by the vehicle determined on the basis of the analysis of the noise signal in connection with a speed analysis of the vehicle.

29. Apparatus for determining a noise signal of a noise source, comprising:
a noise detection system for detecting the noise signal;
a data processing unit for analyzing the noise signal on the basis of signal properties, comparing the noise signal with stored noise patterns, and assigning the noise signal to a noise source type on the basis of the comparison; and
a correction unit, to which the instantaneous position of the noise source is fed.

30. The apparatus as claimed in claim 29, wherein the noise detection system comprises a plurality of noise sensors.

31. The apparatus as claimed in claim 29, wherein the data processing unit comprises a database that includes noise patterns.

32. The apparatus as claimed in claim 29, further comprising a data memory for storing the noise signal.

33. The apparatus as claimed in claim 29, further comprising an optical system for detecting the noise source.

34. The apparatus as claimed in claim 29, further comprising a recording unit for detecting meteorological data.

35. The apparatus as claimed in claim 31, wherein the database includes at least one specific noise pattern for different noise source types in the database.

36. The apparatus as claimed in claim 31, wherein the corrected noise signal can be transmitted to external systems for information.

37. The apparatus as claimed in claim 31, wherein the noise patterns of different noise source types stored in the database have a steady-state, cyclic or transient character.

38. The apparatus as claimed in claim 30, wherein the noise sensors of the noise detection system have a directional characteristic.

39. The apparatus as claimed in claim 29, wherein the data processing unit includes a database of image patterns.

40. A monitoring system comprising the apparatus according to claim 9.

41. A method for classification of a noise source which generates a noise signal, said method comprising:
detecting said noise signal;
determining at least one of position and location dependent ambient conditions of the noise source;
correcting said noise signal based on said at least one of position and ambient conditions of the noise source;
determining properties of said noise signal as corrected;
comparing said noise signal as corrected, with stored noise patterns based on said properties; and
classifying said noise source to a noise source type based on a result of said comparing.

42. Apparatus for classification of a noise source which generates a noise signal, said apparatus comprising:
a noise detection system for detecting the noise signal;
means for determining at least one of position and motion parameters of said noise source;
a correction unit for correcting a detected noise signal based on said at least one of position and motion parameters of said noise source, and generating corrected noise signals;
a data processor for analyzing the corrected noise signals based on selected properties thereof, comparing the corrected noise signals with stored noise patterns based on said properties, and assigning the noise source to a noise source type based on a result of said comparison.
